(19) United States
(12) Patent Application Publication
(10) Pub. No.: US 2018/0161527 A1
(43) Pub. Date: Jun. 14, 2018

(54) EUSTACHIAN TUBE DILATION DEVICE

(71) Applicant: Tyler A. Janz, Punta Gorda, FL (US)

(72) Inventors: Tyler A. Janz, Punta Gorda, FL (US); Karen B. Lu, Walnut, CA (US); Brian Kamradt, Greenwood, IN (US)

(21) Appl. No.: 15/841,086

(22) Filed: Dec. 13, 2017

Related U.S. Application Data
(60) Provisional application No. 62/434,381, filed on Dec. 14, 2016.

(51) Int. Cl. A61M 13/00

(52) U.S. Cl. CPC A61M 13/003; A61M 2205/073; A61M 2205/0216; A61M 2210/0618; A61M 2210/0675

(57) ABSTRACT

A device designed for manual use by patients to help increase the patency of the eustachian tube allowing for pressure equalization and fluid drainage.

This application claims priority under 35 U.S.C. § 119 to U.S. Application Ser. No. 62/434,381, which was filed Dec. 14, 2016 and is expressly incorporated herein by reference.

### TECHNICAL FIELD

The present disclosure relates generally to medical devices and, more specifically, to medical devices for treating eustachian tube dysfunction.

### BACKGROUND

The human ear is composed of three parts: the outer ear, the middle ear, and the inner ear. The outer ear consists of the visible external ear, extending from the external auditory meatus to the tympanic membrane. The middle ear contains ossicles in an air filled cavity, and the inner ear consists of the vestibulocochlear system. The eustachian tube is the “connector” between the middle ear and the posterior portion of the nasopharynx. The eustachian tube is vital for the equalization of pressure between the middle ear and the external environment as well as for the draining of fluid generated by the middle ear through the nasopharynx. At birth, the eustachian tube lies more horizontally and is shorter in length and weaker in strength due to the immaturity of newly formed cartilage in the eustachian canal. By the age of roughly six years old, the eustachian tube should have reached an adult length and consist of stronger cartilage allowing for patency. Children have also been noted to have larger adenoids compared to adults, which can externally compress the eustachian tube preventing ventilation and drainage of the tube.

Eustachian tube dysfunction is a fluid term, which describes a failure for the eustachian tube to allow adequate drainage of fluid or equalization of pressure. One specific type of dysfunction is known as dilatory dysfunction in which the eustachian tube is malfunctioning via several causes including: inflammation due to allergies, anatomic malformation, barotrauma, or congenital anomalies. If there is dysfunction of the eustachian tube, the middle ear becomes a closed system and air is trapped in the middle ear and becomes resorbed thus creating negative pressure in the ear canal. This negative pressure leads to tympanic membrane retraction. Retraction of the tympanic membrane results in loss of optimal tympanic membrane function leading to hearing loss. Furthermore, since the tympanic membrane is highly innervated, irritation from the retraction can lead to pain and discomfort for the patient. Additionally, eustachian tube dysfunction can prevent the proper draining of fluid produced by the middle ear leading to middle ear fluid stasis which can progress to acute or chronic serous otitis media. Children are at a higher risk for developing acute or chronic otitis media and tympanic membrane complications due to the anatomical developmental features of the eustachian tube, causing otitis media to be the most common ear infection seen in children.

Current treatments for eustachian tube dysfunction include decongestants to reduce the production of fluid present in the ear, nose, and throat system and surgical procedures such as tympanostomy tube insertion (external drainage of fluid), eustachian tuboplasty, and cartilage grafting.

Another treatment involves dilating the eustachian tube using a device that applies positive pressure. Such devices include the Earpopper®, which is an electric ear pressure relieving device that is commercially available from Summit Medical and may be used to also relieve inner ear pressure by applying positive pressure while the patient swallows. This technique is often described as the politzerization method. Another device used to relieve pressure is the Eustachi®, which is commercially available from Exercore. Research by several different authors has attempted to quantify the amount of pressure needed to allow the eustachian tube to become patent and allow for the equalization of the middle ear pressure with that of the environment. See Busby D E, SPACE CLINICAL MEDICINE. A PROSPECTIVE LOOK AT MEDICAL PROBLEMS FROM HAZARDS OF SPACE OPERATIONS. Space Life Sci, 1: 157-427(August 1968), 1968:Medium: X; Sss-Otorhinolaryngology And Head & Neck Surgery, Elsevier India Pvt. Limited. Flisberg K IS, Ortengren U. Controlled ear aspiration of air: A physiological test of the Eustachian tube, Acta Otolaryngol Suppl(Stockh), 1963(182):35-38, Dutta N N, Kacker S K, Sinha A. Eustachian tubal function in health and diseases. Indian Journal of Otolaryngology 1971; 23(4):163-175, Srivastava S C, Gupta S C, Singh A P. Efficacy of various methods in evaluation of eustachian tube function, Indian Journal of Otolaryngology and Head and Neck Surgery, 1993; 45(4):188-190, Pandey A, Gupta S, Singh M. A simplified approach to assess variations in Eustachian tubal ventilatory function by Bortnick-Miller apparatus in chronic otitis media cases (dry) before surgery, Indian Journal of Otology, Jul. 1, 2011 2011; 17(3):113-116. Further new data has identified target pressures for temporary eustachian tube dilation. For example, a study by Schröder et. al. noted that 58% of patients diagnosed with chronic eustachian tube (ET) dysfunction experienced a ET opening at 50 mbar, while 94% of healthy patients experienced ET opening at 50 mbar: Clin Otolaryngol, 40(6), 691-697. Furthermore, Mikolajczak et. al's 2014 article “Characterizing the Active Opening of the Eustachian Tube in a Hypobaric/Hyperbaric Pressure Chamber, and Smith et. al.'s 2017 study “Tuboimpedance: A New Test of Eustachian Tube Function” provide further research data regarding eustachian tube dilation pressures

### SUMMARY

According to one aspect, a device designed for manual use by patients to help increase the patency of the eustachian tube allowing for pressure equalization and fluid drainage is disclosed. The device includes a housing sized to be positioned in a hand of a user, a piston received in a chamber defined in the housing, and a tip attached to the housing, the tip having an outlet defined therein. The piston is moveable within the chamber to cause air to advance into and out of the outlet, and when the piston is moved in a first direction within the chamber defined in the housing, pressurized air is advanced in a second direction of the outlet, the second direction being opposite the first direction.

In some embodiments, the device may include a retraction mechanism operable to move the piston in the second direction to cause air to advance into the outlet. In some embodiments, the retraction mechanism may include a biasing element that is configured to move the bias the piston in its fully retracted position to permit air to advance into the outlet.

In some embodiments, the retraction mechanism may include a plurality of teeth coupled to the piston, and a pinion rotatably coupled to the housing and engaged with the plurality of teeth such that when the pinion is rotated in a first rotation direction, the piston is moved in the first direction, and when the pinion is rotated in a second rotation direction, the piston is moved in the second direction.

In some embodiments, the piston may be a first piston, and the medical device may include a second piston that is received in a second chamber defined in the housing. The second piston may be moveable within the chamber and cooperating with the first piston to cause air to advance into and out of the outlet.

In some embodiments, the medical device may include a handle secured to a first end of a shaft. The piston may be attached to a second end of the shaft. In some embodiments, a column may extend outwardly from the housing through the handle to the tip. Additionally, in some embodiments, the handle may include a grip that surrounds the tip such that the tip is positioned between the fingers of a user when the handle is gripped for operation of the device.

According to another aspect, a medical device comprises a housing sized to be positioned in a hand of a user, a piston received in a chamber defined in the housing, and a tip attached to the housing. The tip is sized to be positioned in a patient's nasal cavity and has an outlet defined therein. The piston is moveable within the chamber to cause air to advance into and out of the outlet, and when the piston is moved in a first direction within the chamber defined in the housing, pressurized air is advanced in a second direction of the outlet, the second direction being opposite the first direction. The pressurized air is in a range of 30 to 100 mbar.

In some embodiments, the medical device may further comprise a retraction mechanism operable to move the piston in the second direction to cause air to advance into the outlet.

In some embodiments, the piston may be moveable along the first direction between a retracted position and a depressed position. The retraction mechanism may include a biasing element to bias the piston in the retracted position.

According to another aspect, a medical device comprises a housing sized to be positioned in a hand of a user, a piston received in a chamber defined in the housing, and a tip attached to the housing. The tip is sized to be positioned in a patient's nasal cavity and has an outlet defined therein. The piston is moveable within the chamber to cause air to advance into and out of the outlet, and when the piston is moved in a first direction within the chamber defined in the housing, pressurized air is advanced in a second direction out of the outlet. The second direction is transverse to the first direction, and the pressurized air is in a range of 30 to 100 mbar.

In some embodiments, the piston may be moveable along the first direction between a retracted position and a depressed position, and the retraction mechanism may include a biasing element to bias the piston in the retracted position. In some embodiments, the tip is positioned between a grip defined on the housing such that the tip is positioned between the fingers of a user when the handle is gripped for operation of the device.

According to another aspect, a medical device comprises a housing sized to be positioned in a hand of a user, a handle that is spaced apart from the housing and configured to be engaged by at least one finger of the same hand of the user, a pair of shafts extending from the handle, and a pair of pistons received in chambers defined in the housing. Each piston is secured to an end of one of the pair of shafts. The medical device further comprises a column extending outwardly from the housing through the handle to a tip that is sized to be positioned in a patient's nasal cavity and has an outlet defined therein.

The pistons are moveable within the chamber to cause air to advance into and out of the outlet. When the handle is moved in a first direction toward the housing, the pistons are moved in the first direction within the chambers defined in the housing to advance in a second direction pressurized air along a passageway defined in the column and out from the outlet. The second direction is opposite to the first direction, and the pressurized air is in a range of 30 to 100 mbar.

In some embodiments, the medical device may comprise a retraction mechanism operable to move the piston in the second direction to cause air to advance into the outlet. Additionally, in some embodiments, the retraction mechanism may include a plurality of teeth coupled to the handle, and a pinion rotatably coupled to the housing and engaged with the plurality of teeth such that when the pinion is rotated in a first rotation direction, the handle and the pistons are moved in the first direction, and when the pinion is rotated in a second rotation direction, the handle and the pistons are moved in the second direction.

In some embodiments, the pistons may be moveable along the first direction between a retracted position and a depressed position, and the retraction mechanism includes a biasing element to bias the pistons in the retracted position. In some embodiments, the medical device may also comprise a one-way check valve that permits air to enter the chamber when the piston is moved from a depressed position to a retracted position but retains the air in the chamber.

According to another aspect, a method of dilating an eustachian tube of a patient is disclosed. The method includes gripping a housing of a medical device with a hand to align a tip of the medical device with an opening in a patient's nasal cavity, inserting the tip of the medical device into the opening in the patient's nasal cavity, depressing a handle of the medical device with at least one finger of the hand to move the handle in a first direction away from the tip and toward the housing of the medical device. In the method, depressing the handle causes pressured air in a range of 30 to 100 mbar to advance out of the tip into the patient's nasal cavity in a second direction opposite the first direction.

In some embodiments, the method may further comprise releasing the handle to permit the handle to automatically move in the second direction away from the housing to a retracted position. In the method, movement of the handle in the second direction may draw air into the medical device. Additionally, in some embodiments, the handle may move to the retracted position in between 5 and 10 seconds.

In some embodiments, the method may further comprise operating a pinion on the medical device to move the handle in the second direction away from the housing to a retracted position. In the method, movement of the handle in the second direction may draw air into the medical device.

### BRIEF DESCRIPTION OF THE DRAWINGS

The detailed description particularly refers to the following figures, in which:

FIG. 1 illustrates a perspective view of one embodiment of a medical device for treating eustachian tube dysfunction;

FIG. 2 is a cross-section of the medical device taken along the line 2-2 in FIG. 1;

FIG. 3 is similar to FIG. 1 and illustrates the medical device in a depressed position;

FIG. 4 illustrates a perspective view of another embodiment of a medical device for treating eustachian tube dysfunction;

FIG. 5 is a cross-section taken along the line 5-5 in FIG. 4;

FIG. 6 illustrates a perspective view of another embodiment of a medical device for treating eustachian tube dysfunction; and

FIG. 7 is a cross-section of the medical device of FIG. 6.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

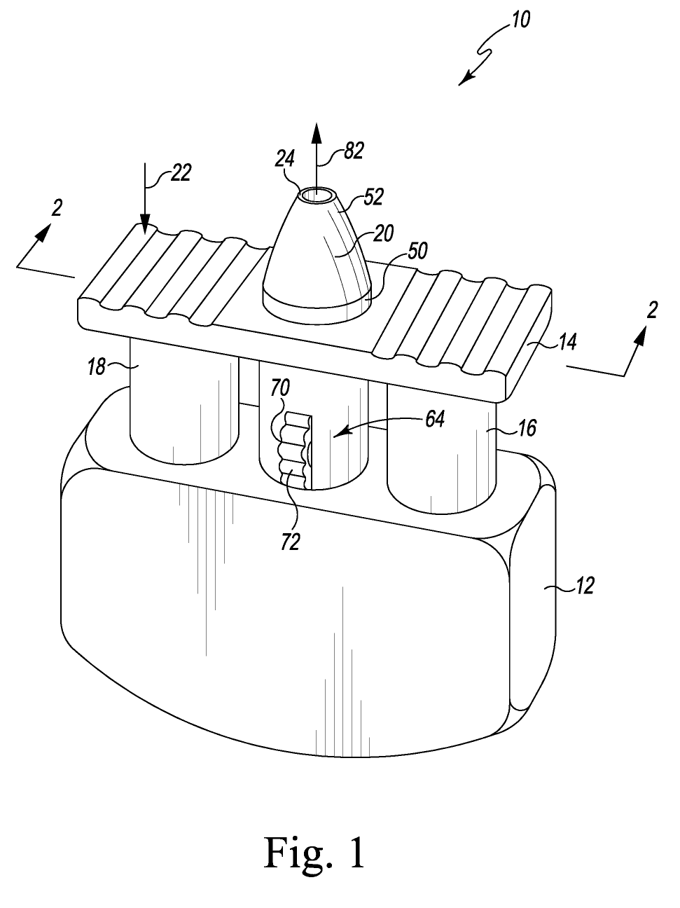

Referring now to FIG. 1, a eustachian tube dilation device 10 is shown. The device 10 includes a housing 12 sized to be gripped by a patient in one hand and a handle 14 moveable relative to the housing 12. In the illustrative embodiment, the housing 12 is configured to fit within the palm of one hand. The device 10 also includes a pair of pumps 16, 18 positioned on either side of a central column 20. To use the device, a patient or other user may position the housing 12 in the palm of one hand and wrap their fingers around the handle 14. In the illustrative embodiment, the central column 20 is configured to be positioned between the fingers of the patient when the handle is gripped for operation of the device. The patient may then tighten their fingers, causing the handle 14 to move in the direction indicated by arrow 22 and thereby depressing the pumps 16, 18. As described in greater detail below, the movement of the pumps 16, 18 causes pressurized air to advance outward from an outlet 24 defined in the central column 20.

In the illustrative embodiment, the housing 12, the handle 14, and the column 20 may be formed from medical grade polymeric or metallic materials. In some embodiments, the housing 12 and the column 20 may be formed as a single monolithic component.

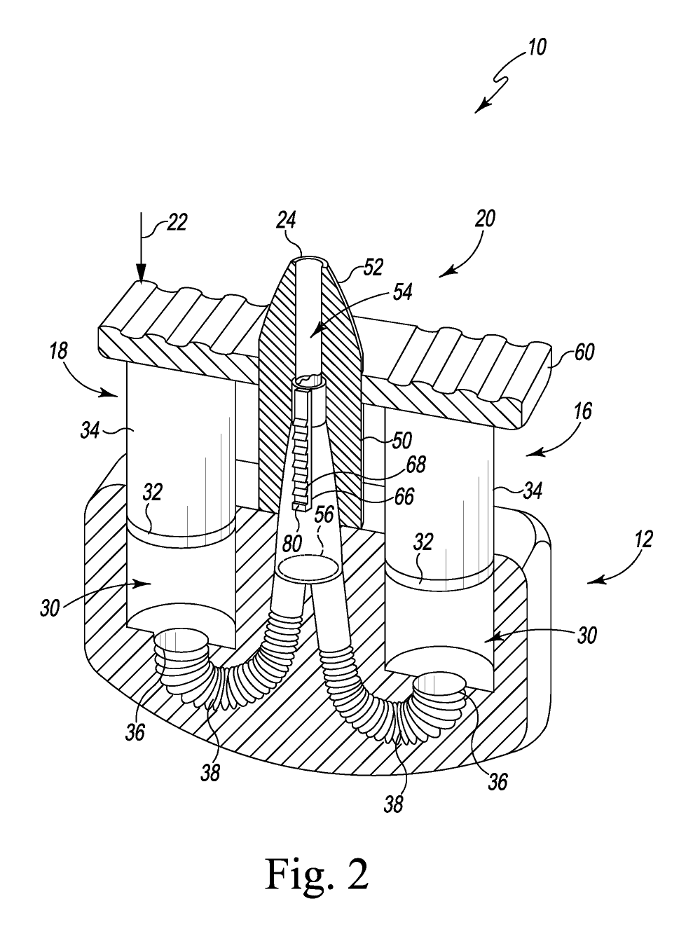

As shown in FIG. 2, each of the pumps 16, 18 includes a chamber or passageway 30 defined in the housing 12 and a piston 32 that is positioned in the passageway 30. A shaft 34 extends between each piston 32 and the handle 14. Each piston 32 includes a seal (not shown) that engages the cylindrical walls lining each passageway 30 to prevent the passage of air from the passageways 30 to the shafts 34. Each pump 16, 18 further includes a port 36 that is positioned at the base of the passageway 30. A hose or tube 38 is connected to each port 36. In the illustrative embodiment, the pistons 32, shafts 34, and hoses 38 may be formed from medical grade polymeric or metallic materials. In some embodiments, each piston 32 may be formed with a corresponding shaft 34 as a single monolithic component. In other embodiments the device may include only a single pump. In some embodiments, the hoses may be integrated into the housing.

The central column 20 includes a main shaft 50 that extends outwardly from the housing 12 to a tip 52 sized to be positioned in a patient's nasal cavity. In the illustrative embodiment, the tip 52 is conical or dome-shaped and is configured to be positioned between the fingers of the patient when the handle is gripped for operation of the device. The outlet 24 is defined in the tip 52, as shown in FIG. 2. A column passageway 54 extends from the outlet 24 to an inlet 56 positioned in the housing 12. In the illustrative embodiment, the hoses 38 extend between the ports 36 and the inlet 56. It should be appreciated that in other embodiments the housing 12 may have additional passageways defined therein such that one or both of the hoses may be eliminated.

The handle 14 includes an elongated plate 60 sized to be gripped by the fingers of the patient. It should be appreciated that the plate 60 may be, for example, contoured or knurled to assist the patient in gripping the plate 60. In the illustrative embodiment, the central column 20 extends through an opening 62 defined in the plate 60 and is positioned between the fingers of the patient or user when the handle is gripped for operation of the device.

The device 10 also includes a retraction mechanism 64 that may be operated by the patient or other user to move the pumps 16, 18 to the retracted position shown in FIGS. 1-2. In the illustrative embodiment, the mechanism 64 includes a bar 66 that extends downwardly from the handle 14. The bar 66 includes a plurality of teeth 68. As shown in FIG. 1, the retraction mechanism 64 also includes a wheel or pinion 70 pivotally coupled to the central column 20. The pinion 70 includes a plurality of teeth 72 that are interdigitated with the teeth 68 of the bar 66 such that when the pinion 70 is rotated, the bar 66 (and hence the handle 14) is moved toward or away from the housing 12 of the device 10. It should be appreciated that the pinion and bar may be located on the housing. Additionally, it should be appreciated that in other embodiments the retraction mechanism may take other forms, including, for example, an electrical actuator (such as a motor) to push the handle 14 outward. In still other embodiments, the retraction mechanism may be omitted and a patient may pull the handle 14 outward while holding onto the housing 12. In the illustrative embodiment, the bar 66 also includes a tab or latch 80 configured to engage a flange or lip (not shown) formed in the housing 12 to limit movement of the handle 14 away from the housing 12. The pinion 70 and the bar 66 may be formed from medical grade polymeric or metallic materials.

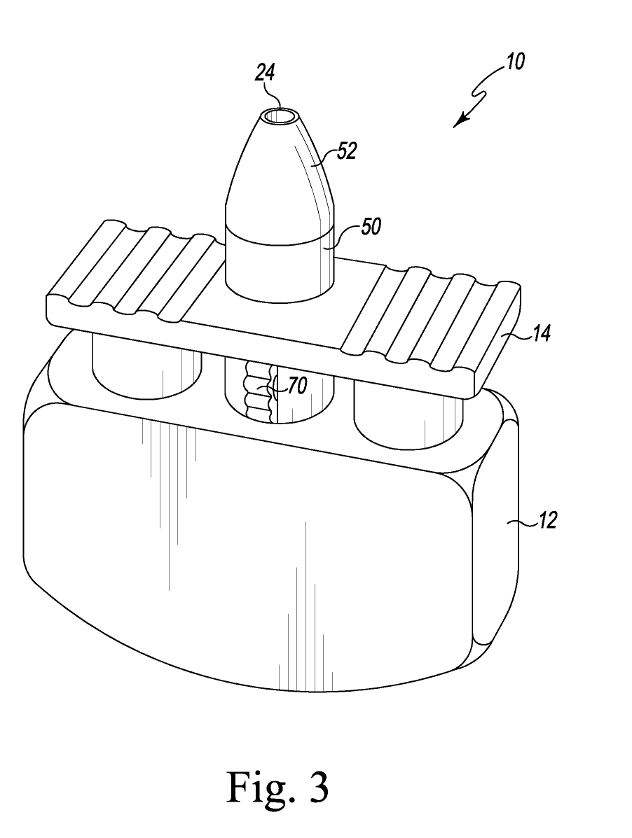

The device 10 is designed for manual use by patients or other users to help increase the patency of the eustachian tube allowing for pressure equalization and fluid drainage. The device 10 is a closed pressure system that generates positive pressure via the pumps 16, 18. The patient will retract the pumps 16, 18 from the position shown in FIG. 3 to the position shown in FIG. 1 using the pinion wheel 70, which functions to raise the pistons 32 within the passageways 30 to generate pressurized air in the two pumps 16, 18. The interaction between the latch 80 and the housing 12 limits the amount of pressure that can be generated and also prevents separation of pumps 16, 18 and the handle 14 from the housing 12. It should be appreciated that in other embodiments, the device 10 may include a one-way check valve to retain pressurized air within the passageways 30.

As the handle 14 is moved in the direction indicated by arrow 22 in FIG. 1, air is pushed from the two pumps 16, 18, through the hoses 38, and into the column passageway 54. The pressurized air is advanced along the passageway 54 and out through the outlet 24 in the direction indicated by arrow 82 in FIG. 1. When using the device 10, the patient should optionally have the device 10 in one hand while compressing the other nostril with the other hand. This will create a closed pressure system for the device 10. As the handle 14 is depressed, the patient should perform the Lowry method (Valsalva and Toynbee) to open the eustachian tube to allow the air to flow and open/clear the eustachian tube.

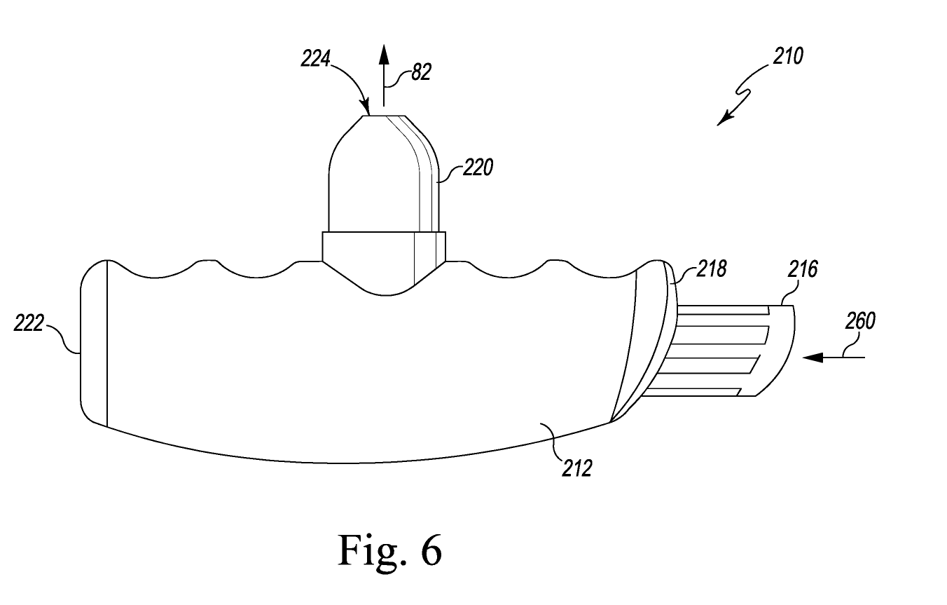

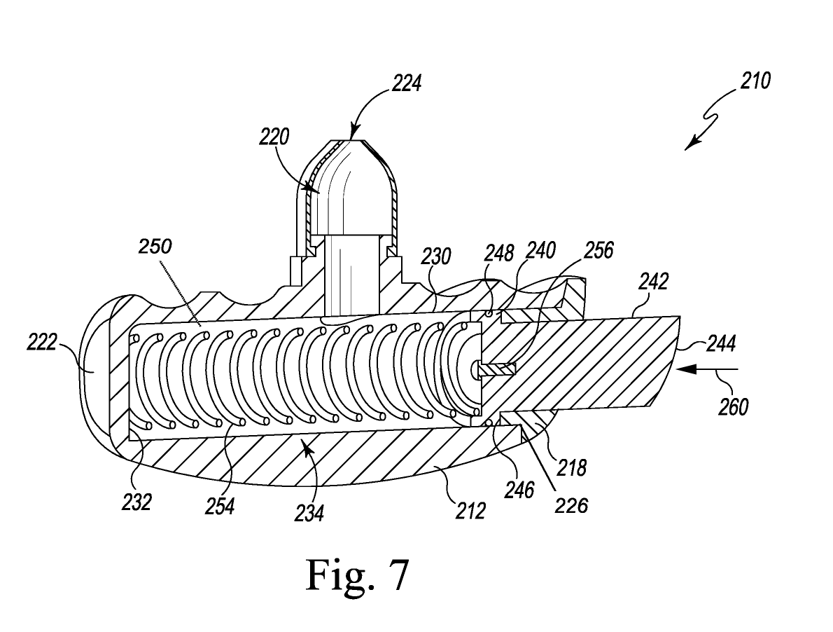

In the illustrative embodiment, the pistons 32, passageways, and outlet are sized such that the device 10 generates a positive pressure within the range of 20 to 30 mm Hg (25 mbar to 40 mbar). In some embodiments, the device 10 generates a positive pressure within the range of 30 mbar to 100 mbar or within a range of 50 mbar to 100 mbar. Additionally, in use, the bar 66 is sized to permit about five to ten seconds of movement of the pistons 32 between the retracted and depressed positions to allow the patient to close the opposite nostril with his or her other hand and also depress the apparatus. It should be appreciated that in other embodiments the components of the device may be resized or altered to produce different amounts of positive pressure at different rates. In some embodiments such as, for example, the embodiment shown in FIGS. 6-7, the device may include only a single piston to generate positive pressure.

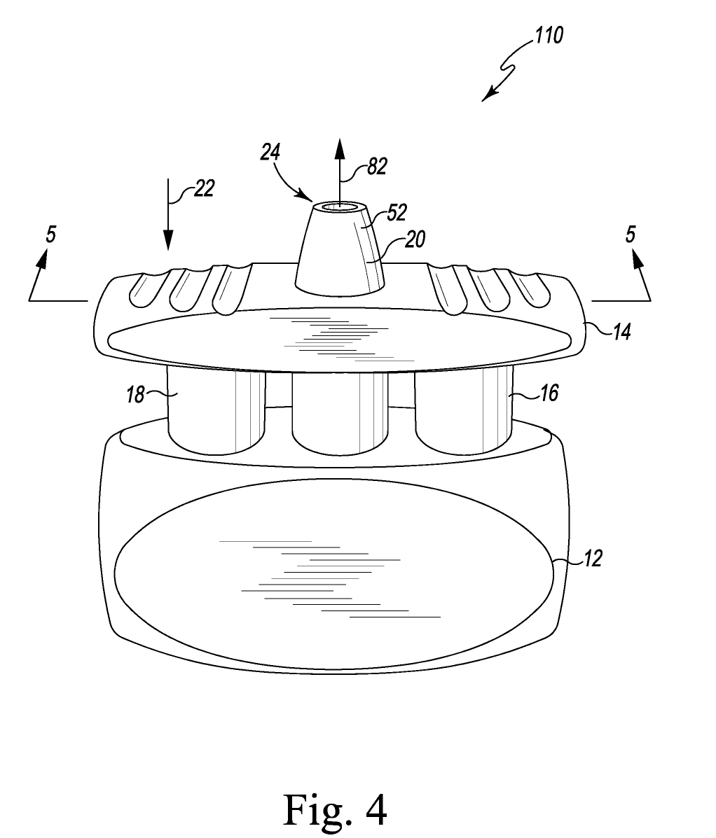

Referring now to FIG. 4, another embodiment of a eustachian tube dilation device (hereinafter device 110) is shown. The device 110 includes features similar to the device 10. The reference numbers used to describe features in the device 10 will be used to describe features that are the same or similar in the device 110. The device 110 includes a housing 12 sized to be gripped by a patient in one hand and a handle 14 moveable relative to the housing 12. The device 10 also includes a pair of pumps 16, 18 positioned on either side of a central column 20. To use the device, a patient or other user may position the housing 12 in the palm of one hand and wrap their fingers around the handle 14. The patient may then tighten their fingers, causing the handle 14 to move in the direction indicated by arrow 22 and thereby depressing the pumps 16, 18. As described in greater detail below, the movement of the pumps 16, 18 causes pressurized air to advance outward from an outlet 24 defined in the central column 20.

In the illustrative embodiment, the housing 12, the handle 14, and the column 20 may be formed from medical grade polymeric or metallic materials. In some embodiments, the housing 12 and the column 20 may be formed as a single monolithic component.

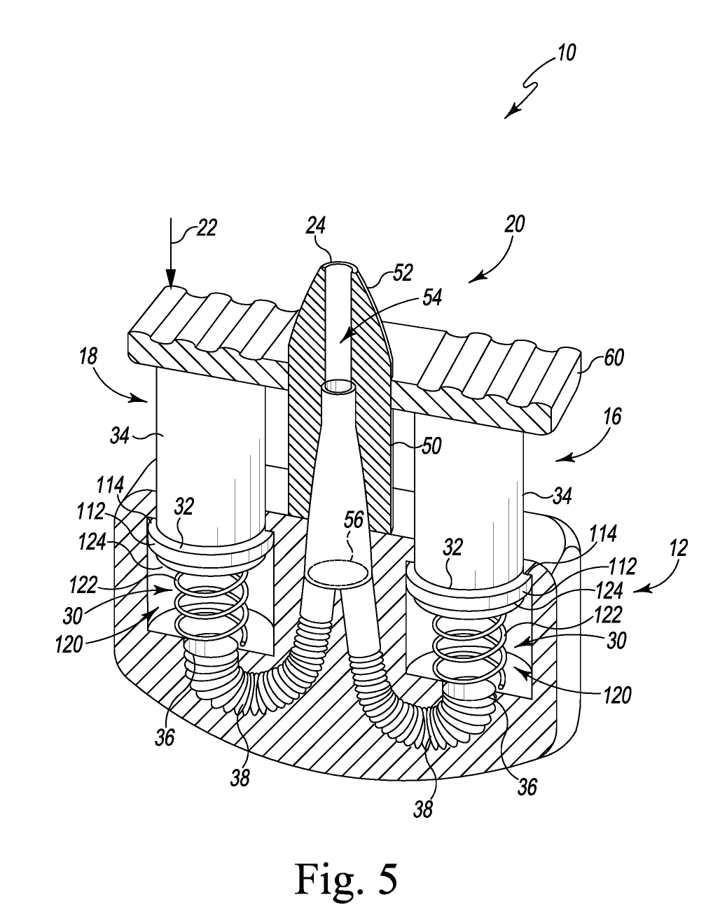

As shown in FIG. 5, each of the pumps 16, 18 includes a passageway 30 defined in the housing 12 and a piston 32 that is positioned in the passageway 30. A shaft 34 extends between each piston 32 and the handle 14. Each piston 32 includes a seal (not shown) that engages the cylindrical walls lining each passageway 30 to prevent the passage of air from the passageways 30 to the shafts 34. Each piston 32 also includes an annular flange 112 that engages an inner wall 114 of the housing 12 to retain the pistons 32 in the passageways 30. Each pump 16, 18 further includes a port 36 that is positioned at the base of the passageway 30 as well as a reload spring 48. A hose or tube 38 is connected to each port 36. In the illustrative embodiment, the pistons 32, shafts 34, and hoses 38 may be formed from medical grade polymeric or metallic materials. In some embodiments, each piston 32 may be formed with a corresponding shaft 34 as a single monolithic component. In other embodiments the device may include only a single pump.

The central column 20 includes a main shaft 50 that extends outwardly from the housing 12 to a tip 52 sized to be positioned in a patient's nasal cavity. In the illustrative embodiment, the tip 52 is conical or dome-shaped. The outlet 24 is defined in the tip 52, as shown in FIG. 5. A column passageway 54 extends from the outlet 24 to an inlet 56 positioned in the housing 12. In the illustrative embodiment, the hoses 38 extend between the ports 36 and the inlet 56. It should be appreciated that in other embodiments the housing 12 may have additional passageways defined therein such that one or both of the hoses may be eliminated.

The handle 14 includes an elongated plate 60 sized to be gripped by the fingers of the patient. It should be appreciated that the plate 60 may be, for example, contoured or knurled to assist the patient in gripping the plate 60. In the illustrative embodiment, the central column 20 extends through an opening 62 defined in the plate 60.

The device 110 also includes a retraction mechanism 120 to move the pumps 16, 18 to the retracted position shown in FIGS. 4 and 5. As shown in FIG. 5, the retraction mechanism 120 includes a biasing element such as, for example, a pair of springs 122, which are configured to bias each piston 32 in the retracted position. The retraction mechanism 120 also includes a valve spring retainer 124 that is secured to each piston 32 to provide seats for the springs 122. When a patient or other user applies force on the handle 14 in the direction indicated by arrow 22, the bias exerted by the springs 122 is overcome, thereby permitting the pistons 32 to move along the passageways 30 and force air through the hoses 38 and into the column passageway 54. When the user releases the handle 14, the springs 122 urge the pistons 32 upward and the handle 14 outward to place the pistons 32 back in contact with the inner walls 114 of the housing 12. In other words, once the handle 14 is let go, the pistons 32 will retract back to their starting position via the force exerted by the springs 122, thereby drawing air into the passageways 30. It should be appreciated that in other embodiments the device 110 may include a one-way check valve to retain pressurized air within the passageways 30. It should also be appreciated that in other embodiments the retraction mechanism may take other forms, including, for example, an electrical actuator (such as a motor) to push the handle 14 outward. In still other embodiments, the retraction mechanism may be omitted and a patient may pull the handle 14 outward while holding onto the housing 12.

The device 110 is designed for manual use by patients to help increase the patency of the eustachian tube allowing for pressure equalization and fluid drainage. The device 110 is a closed pressure system that generates positive pressure via the pumps 16, 18. As the handle 14 is moved in the direction indicated by arrow 22 in FIG. 1, air is pushed from the two pumps 16, 18, through the hoses 38, and into the column passageway 54. The pressurized air is advanced along the passageway 54 and out through the outlet 24 in the direction indicated by arrow 82 in FIG. 4.

In the illustrative embodiment, the pistons 32, passageways, and outlet are sized such that the device 110 generates a positive pressure within the range of 20 to 30 mm Hg (25 mbar to 40 mbar). In some embodiments, the device 110 generates a positive pressure within the range of 30 mbar to 100 mbar or within a range of 50 mbar to 100 mbar. Additionally, in use, the springs 122 are sized to permit about five to ten seconds of movement of the pistons 32 between the retracted and depressed positions to allow the patient to close the opposite nostril with his or her other hand and also depress the apparatus.

Referring now to FIGS. 6-7, another embodiment of a eustachian tube dilation device (hereinafter dilation device 210) is shown. The dilation device 210 includes a housing 212 that is sized to be gripped by a patient in one hand and a plunger 216 that extends outwardly from an end 218 of the housing 212. In the illustrative embodiment, the housing 212 is configured to fit within the palm of one hand. A nose piece 220 is also attached to the housing 212 between the end 218 and the opposite end 222 of the housing 212. The nose piece 220 is positioned on the housing 212 such that the hose piece 220 is positioned between the fingers of the patient or other user when gripping the housing 212 for use. To use the device, a patient or other user may position the housing 212 in the palm of one hand and engage the plunger 216 with one finger. The user may then depress the plunger 216 to cause pressurized air to advance outward from an outlet 224 of the nose piece 220, as described in greater detail below.

In the illustrative embodiment, the housing 212 and the plunger 216 are formed from medical grade polymeric or metallic materials. The nose piece 220 is formed from a medical grade elastomeric material such as, for example, silicone.

As shown in FIG. 7, the housing 212 has an opening 226 that is defined in the end 218. An inner wall 230 extends inwardly from the opening 226 to a base wall 232 positioned at the opposite end 222 of the housing 212. The inner wall 230 and the base wall 232 cooperate to define a passageway 234 and the housing 212.

The plunger 216 includes a piston 240 that is positioned in the passageway 234 of the housing 212 and an elongated body 242 that extends from the piston 240 to a distal tip 244 positioned outside of the housing 212. As shown in FIG. 7, the inner wall 230 of the housing 212 includes a rim surface 246 that is configured to be engaged by the piston 240 of the plunger 216 such that the piston 240 is retained in the passageway 234. The plunger 216 also includes a seal 248 that engages the inner wall 230 to prevent air in the passageway 234 from exiting through the opening 226 of the housing 212.

The device 210 also includes a retraction mechanism 250 to move the plunger 216 to the retracted position shown in FIGS. 6 and 7. In the retracted position, the piston 240 is engaged with the rim surface 246 of the housing 212. As shown in FIG. 7, the retraction mechanism 250 includes a biasing element such as, for example, a spring 254, which is configured to bias the piston 240 into engagement with the rim surface 246. The retraction mechanism 250 also includes a spring retainer 256 that is secured to piston 240 to provide a seat for the spring 254. When a patient or other user applies force to the distal tip 244 of the plunger 216 in the direction indicated by arrow 260, the bias exerted by the spring 254 is overcome thereby permitting the piston 240 to move along the passageway 234 and force air out of the outlet 224 of the nose piece 220. The device 210, like the devices 10, 110 described above, is therefore designed for manual use by patients to help increase the patency of the eustachian tube allowing for pressure equalization and fluid drainage. As a closed pressure system that generates positive pressure, the device 210 permits pressurized air to advance along the passageway 234 and out through the outlet 224 in the direction indicated by arrow 82 in FIG. 6.

When the user releases the distal tip 244, the spring 254 urges the plunger 216 outward to place the piston 240 back into contact with the rim surface 246 of the housing 212. This movement of the piston 240 back to the retracted position draws external air into the outlet 224 to “rearm” the device 210 for additional use. It should be appreciated that in other embodiments the device 210 may include a one-way check valve to retain pressurized air within the passageways 30. It should also be appreciated that in other embodiments the retraction mechanism may take other forms including, for example, elastomeric polymer, electrical actuator, or other device capable of drawing the plunger 216 back to the retracted position. In still other embodiments, the retraction mechanism may be omitted, and a patient may pull the plunger 216 manually outward while holding onto the housing 212.

In the illustrative embodiment, the piston 240 and outlet are sized such that the device 210 generates a positive pressure within the range of 20 to 30 mm Hg (25 mbar to 40 mbar). In some embodiments, the device 210 generates a positive pressure within the range of 30 mbar to 100 mbar or within a range of 50 mbar to 100 mbar. Additionally, in use, the spring 254 is sized to permit about five to ten seconds of movement of the piston 240 between the retracted and depressed positions to allow the patient to close the opposite nostril with his or her other hand and also depress the apparatus

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A medical device, comprising:
a housing sized to be positioned in a hand of a user,
a piston received in a chamber defined in the housing, and
a tip attached to the housing, the tip being sized to be positioned in a patient's nasal cavity and having an outlet defined therein,
wherein the piston is moveable within the chamber to cause air to advance into and out of the outlet, and when the piston is moved in a first direction within the chamber defined in the housing, pressurized air is advanced in a second direction of the outlet, the second direction being opposite the first direction, wherein the pressurized air is in a range of 30 to 100 mbar.

2. The medical device of claim 1, further comprising a retraction mechanism operable to move the piston in the second direction to cause air to advance into the outlet.

3. The medical device of claim 2, wherein the retraction mechanism includes:
a plurality of teeth coupled to the piston, and
a pinion rotatably coupled to the housing and engaged with the plurality of teeth such that (i) when the pinion is rotated in a first rotation direction, the piston is moved in the first direction, and (ii) when the pinion is rotated in a second rotation direction, the piston is moved in the second direction.

4. The medical device of claim 2, wherein:
the piston is moveable along the first direction between a retracted position and a depressed position, and
the retraction mechanism includes a biasing element to bias the piston in the retracted position.

5. The medical device of claim 1, wherein the piston is a first piston, and the medical device includes a second piston that is received in a second chamber defined in the housing, the second piston being moveable within the chamber and cooperating with the first piston to cause air to advance into and out of the outlet.

6. The medical device of claim 1, further comprising a handle secured to a first end of a shaft, wherein the piston is attached to a second end of the shaft.

7. The medical device of claim 6, wherein a column extends outwardly from the housing through the handle to the tip.

8. A medical device, comprising:
a housing sized to be positioned in a hand of a user,
a piston received in a chamber defined in the housing, and
a tip attached to the housing, the tip being sized to be positioned in a patient's nasal cavity and having an outlet defined therein,
wherein the piston is moveable within the chamber to cause air to advance into and out of the outlet, and when the piston is moved in a first direction within the chamber defined in the housing, pressurized air is advanced in a second direction of the outlet, the second direction being transverse to the first direction, wherein the pressurized air is in a range of 30 to 100 mbar.

9. The medical device of claim 8, wherein:
the piston is moveable along the first direction between a retracted position and a depressed position, and
the retraction mechanism includes a biasing element to bias the piston in the retracted position.

10. A medical device, comprising:
a housing sized to be positioned in a hand of a user,
a handle that is spaced apart from the housing and configured to be engaged by at least one finger of the hand of the user,
a pair of shafts extending from the handle,
a pair of pistons received in chambers defined in the housing, each piston being secured to an end of one of the pair of shafts, and
a column extending outwardly from the housing through the handle to a tip that is sized to be positioned in a patient's nasal cavity and has an outlet defined therein,
wherein the pistons are moveable within the chambers to cause air to advance into and out of the outlet, and
wherein when the handle is moved in a first direction toward the housing, the pistons are moved in the first direction within the chambers defined in the housing to advance in a second direction pressurized air along a passageway defined in the column and out from the outlet, the second direction being opposite to the first direction, wherein the pressurized air is in a range of 30 to 100 mbar.

11. The medical device of claim 10, further comprising a retraction mechanism operable to move the piston in the second direction to cause air to advance into the outlet.

12. The medical device of claim 11, wherein the retraction mechanism includes:
a plurality of teeth coupled to the handle, and
a pinion rotatably coupled to the housing and engaged with the plurality of teeth such that (i) when the pinion is rotated in a first rotation direction, the handle and the pistons are moved in the first direction, and (ii) when the pinion is rotated in a second rotation direction, the handle and the pistons are moved in the second direction.

13. The medical device of claim 12, wherein:
the pistons are moveable along the first direction between a retracted position and a depressed position, and
the retraction mechanism includes a biasing element to bias the pistons in the retracted position.

14. A method of dilating an eustachian tube of a patient, comprising:
gripping a housing of a medical device with a hand to align a tip of the medical device with an opening in a patient's nasal cavity,
inserting the tip of the medical device into the opening in the patient's nasal cavity,
depressing a handle of the medical device with at least one finger of the hand to move the handle in a first direction away from the tip and toward the housing of the medical device,
wherein depressing the handle causes pressured air in a range of 30 to 100 mbar to advance out of the tip into the patient's nasal cavity in a second direction opposite the first direction.

15. The method of claim 14, further comprising:
releasing the handle to permit the handle to automatically move in the second direction away from the housing to a retracted position,
wherein movement of the handle in the second direction draws air into the medical device.

16. The method of claim 15, wherein the handle moves to the retracted position in between 5 and 10 seconds.

17. The method of claim 14, further comprising:
operating a pinion on the medical device to move the handle in the second direction away from the housing to a retracted position,
wherein movement of the handle in the second direction draws air into the medical device.
